(19) United States
(12) Patent Application Publication
(10) Pub. No.: US 2010/0293843 A1
(43) Pub. Date: Nov. 25, 2010

(54) METHOD OF PREPARING ALCOHOL ESTERS FROM TRIGLYCERIDES AND ALCOHOLS USING HETEROGENEOUS CATALYSTS BASED ON A HYBRID SOLID WITH AN ORGANIC-INORGANIC MIXED MATRIX

(75) Inventors: Delphine Bazer-Bachi, Saint-Genis-Laval (FR); Vincent Lecocq, Brignais (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: IFP, RUEIL-MALMAISON CEDEX (FR)

(21) Appl. No.: 12/680,115

(22) PCT Filed: Sep. 25, 2008
(86) PCT No.: PCT/FR08/01330

§ 371 Date: Aug. 3, 2010

(30) Foreign Application Priority Data
Sep. 28, 2007 (FR) 07/06852
Sep. 28, 2007 (FR) 07/06853

(51) Int. Cl. C10L 1/19; C07C 67/00

(52) U.S. Cl. 44/398; 554/124

(57) ABSTRACT

A method of preparing a composition of alcohol esters of linear monocarboxylic acids with 6 to 26 carbon atoms from a vegetable or animal oil, neutral or acid, virgin or recycled, with monoalcohols having 1 to 18 carbon atoms, in the presence of a heterogeneous catalyst based on a hybrid solid with an organic-inorganic mixed matrix, allows to directly produce, in one or more stages, an ester that can be used as fuel and a pure glycerin.

### FIELD OF THE INVENTION

The present invention relates to a new method of preparing alcohol esters of monocarboxylic acids from fatty substances of vegetable or animal origin.

The mainly desired reaction is a transesterification carried out according to path I below and possibly a coupled esterification and transesterification reaction, esterification being achieved according to path II below.

1 triglyceride+3 alcohols→3 fatty substance esters+glycerin Path I

Fatty acid+alcohol→fatty acid esters+water

Fatty acid+glycerin→glyceride+water Path II

### BACKGROUND OF THE INVENTION

Fatty substance esters are currently used in many applications as diesel fuels, furnace fuel oils, ecological solvents, base compounds for manufacturing fatty alcohol sulfonates, amides, ester dimers, etc.

In the case of diesel fuel, which is today a major application for fatty substance esters, a certain number of specifications have been established, whose list, limits and methods belong to standard EN 14,214 (2003) currently applicable in Europe. The ester must contain at least 96.5 mass % esters, at most 0.8 mass % monoglycerides, at most 0.2 mass % diglycerides and at most 0.2 mass % triglycerides, few free fatty acids (<0.5 mg KOH per g) that may be corrosive, less than 0.25 mass % bonded and free glycerin, and metals only as traces. This involves a precise protocol to obtain the desired purity.

When preparing an ester from oil or fat and monoalcohol, depending on the nature of the oil initially used, 10 to 15 mass % of a secondary product, which is glycerin, automatically forms. This glycerin is sold at a high price for various uses, but only when it is of high purity, which is obtained after advanced purification in units specialized in vacuum distillation.

In short, most commercial ester manufacturing methods lead quite readily to raw products (esters and glycerin) that however have to be deeply purified using various treatments that eventually burden the conversion cost.

It is well known to produce methyl esters using conventional means such as homogeneous catalysis with soluble catalysts, such as soda or sodium methylate, by reacting a neutral oil and an alcohol such as methanol (for example JAOCS 61, 343-348 (1984)). A pure product that can be used as fuel and a glycerin meeting the specifications are however obtained only after many stages. In fact, the glycerin obtained is polluted by alkaline salts or alcoholates, so that the glycerin purification plant is almost as costly as the ester manufacturing plant.

Heterogeneous catalysis methods afford the advantage of producing catalyst-free esters and glycerin, which are therefore easily purified. However, it is often difficult to economically obtain both an ester and a glycerin of high purity.

European patent EP-B-0,198,243 describes the manufacture of methyl esters by transesterification of an oil with methanol, using as the catalyst an alumina or a mixture of alumina and of ferrous oxide. However, the LHSV (volume of oil injected/volume of catalyst/hour) is low, the amount of glycerin collected is much less than that theoretically expected and the purity of the esters obtained is rather low (ranging between 93.5% and 98%).

Methods using a catalytic system based on metallic oxides, alone or in combination, deposited or not on an alumina, have been described. Patent FR-B-2,752,242 filed by the applicant describes the use of solid and non soluble catalysts formed from alumina and zinc oxide or zinc aluminate. Patent applications EP-A-1,505,048 and EP-A-1,593,732, also filed by the applicant, describe a vegetable or animal oil transesterification method using heterogeneous catalysts based on mixtures of alumina and titanium oxide, alumina and zirconium oxide, alumina and antimony oxide, or combinations of titanium and zinc oxides, of alumina, titanium and zinc oxides, of titanium and bismuth oxides or of alumina, titanium and bismuth oxides.

Besides these oxide type solids, an increasing number of new base phases has been used for catalyzing the transesterification of oils with alcohols.

By way of example, De Filippis et al. (Energy & Fuels 2005, 19, 225-228) suggest using sodium phosphate to catalyze the rapeseed oil transesterification reaction.

Suppes et al. (Applied Catalysis A: general 257 (2004) 213-223) use many different materials such as Cs or K-exchanged zeolites or metals that make up the reactors, for soybean oil transesterification.

### SUMMARY OF THE INVENTION

The present invention describes a method of preparing a composition of alcohol esters of linear monocarboxylic acids with 6 to 26 carbon atoms and glycerin wherein a fatty substance of animal or vegetable origin is reacted with an aliphatic monoalcohol having 1 to 18 carbon atoms, in the presence of at least one heterogeneous catalyst based on a hybrid solid with an organic-inorganic mixed matrix.

These porous hybrid solids with an organic-inorganic mixed matrix are coordination polymers. They consist of metal ions or of metal ion polyhedra associated with one another by at least one at least bidentate polyfunctionalized organic ligand.

Organic-inorganic hybrid solids based on metals connected to each other by organic molecules can be used for applications such as gas storage, hydrogen storage for example (U.S. Pat. No. 7,196,210; Yaghi, J. Am. Chem. Soc., 127, 17998; Zhou, J. Am. Chem. Soc., 128, 3896).

Catalysis applications of these materials are much rarer. However, they have been used for reactions such as alcoxylation (U.S. Pat. No. 7,202,385), epoxidation (U.S. Pat. No. 6,624,318), asymmetric aldehyde alkylation (Lin, J., Am. Chem. Soc., 2005, 127, 8940), cyanosylilation (Fujita, Chem. Commun., 2004, 1586). Very recently, Llabrès et al. (Journal of Catalysis, 250 (2007) 294-298) showed the activity of a hybrid palladium material for alcohol oxidation, Suzuki coupling and olefin hydrogenation reactions.

A material based on the element zinc and on a chiral pyridinic ligand has been synthesized by Kim et al. to catalyze the enantioselective transesterification of 2,4-dinitrophenyl acetate by an alcohol. However, this material, whose synthesis is complex, is poorly active because the conversion reaches 90% only after about one hundred hours reaction with, furthermore, extremely low enantiomeric excesses (below 10%) (Kim, Nature, 404, 2000, 982). This reaction involves an ester activated by electroattractor nitro groups, in the presence of a solvent at ambient temperature. Using activated monoesters, whose steric hindrance is furthermore low, makes a fundamental difference with the transesterification of triglycerides or fatty acid triesters, which takes place at higher temperatures according to a mechanism consisting of consecutive reactions involving fatty acid derivatives that all have a high steric hindrance. Besides, the transesterification reaction of the fatty substances occurs in the absence of a solvent. All of these parameters (absence of solvent, high temperature, sterically hindered reagents of different natures) significantly distinguishes the transesterification of fatty substances from an enantioselective transesterification. Thus, according to the results provided by Kim et al., it appears that using a functionalized hybrid solid whose synthesis is complex is of minor interest for ester conversion reactions. Besides, the small pore size of these solids, as well as the absence of chemical functions in the framework of the material for the simplest, did not predestine these coordination polymers to be used as catalysts in reactions involving fatty substances.

Surprisingly enough, we have shown that catalysts based on porous hybrid solids with an organic-inorganic mixed matrix advantageously have the capacity of catalyzing the transesterification of fatty substances with methanol and with heavier alcohols. Thus, it is possible to form ethyl, isopropyl or butyl esters that are of interest because the flow points of esters formed with ethyl, isopropyl or butyl alcohols are often lower than those of methyl esters, the gain being sometimes 10° C., which allows to initially use more saturated oils.

One advantage of the invention using a catalyst based on porous hybrid solids with an organic-inorganic matrix is notably to allow a decrease in the reaction temperature, the contact time between the reagents or the alcohol/fatty substance ratio in relation to the prior art, while improving the conversion rate and maintaining a high ester selectivity.

Another advantage of the invention lies in the fact that these solids catalyze transesterification and esterification reactions according to a heterogeneous catalysis process. Thus, the catalyst is not consumed in the reaction and is not dissolved in the reaction medium. By remaining in the solid form, it is easily separated from the reaction medium without catalyst loss and without pollution of the reaction medium by dissolved species or catalyst residues.

The activity and the selectivity of this catalyst are not affected by the transesterification or esterification reaction: the catalyst is stable and recyclable under the experimental reaction conditions. This type of catalyst is compatible with use in a continuous industrial process, with a fixed bed for example, wherein the catalyst feed can be used for a very long time without any activity loss.

The method according to the invention is described more in detail hereafter.

### DETAILED DESCRIPTION

### Fatty Substances

The fatty substances used in the method according to the invention correspond to natural or elaborate substances, of animal or vegetable origin, predominantly containing triglycerides, commonly referred to as oil and fats.

Examples of oils that can be used are all the common oils, such as palm oil (concrete or olein), soybean oil, palm nut oil, copra oil, babassu oil, rapeseed oil (old or new), sunflower oil (conventional or oleic), corn oil, cotton oil, peanut oil, pourgher oil (Jatropha curcas), castor oil, linseed oil and crambe oil, and all the oils obtained from sunflower and rapeseed for example by genetic engineering or hybridization, or obtained from algae.

It is even possible to use waste kitchen oil, slaughterhouse oil, various animal oils such as fish oil, seal oil, tallow, lard, fat from sewage treatment and even fowl fat, because the esters manufactured from some alcohols such as ethyl, isopropyl or butyl alcohol allow to gain more than 10° C. in pour point and consequently to initially use more saturated oils.

The oils used can also include partly modified oils, for example by polymerization or oligomerization, such as for example linseed oil or sunflower oil “stand oils”, and blown vegetable oils.

The oils used are neutral or acid, virgin or recycled oils.

The presence of fatty acids in the oils is not a priori harmful because catalytic systems based on porous hybrid solids with an organic-inorganic mixed matrix are also active for esterification and they also convert fatty acids to esters. The limit value for free fatty acids contained in the oils is an acid number close to 10 (the acid number being defined as the mass in mg of KOH required to titrate all the free fatty acids in 1 g oil). The operability of the method under such conditions is close to that defined with an oil having a low acid number (i.e. below 0.2 mg KOH/g).

In the case of oils with a very high acid number (close to 10 mg KOH/g), one option consists in preceding the transesterification reaction by an esterification reaction of the free fatty acids present, using either the same alcohol as the alcohol used in the transesterification method in the presence of a strong acid such as sulfuric acid or soluble or supported sulfonic acids (of Amberlyst 15® resins type), or using preferably glycerin, to form a total or partial glycerol ester, using the same catalytist based on porous hybrid solids with an organic-inorganic mixed matrix, at atmospheric pressure and preferably under vacuum, and at temperatures ranging between 150° C. and 220° C.

When using waste kitchen oils, which are a very cheap raw product for the production of a biodiesel fuel, the fatty acid polymers have to be removed from the reaction mixture so that the mixture of esters meets the specifications of the EN 14214 standard.

Alcohol

The nature of the alcohol used in the method plays a part in the transesterification activity.

In general terms, it is possible to use various aliphatic monoalcohols having for example 1 to 18 carbon atoms, preferably 1 to 12 carbon atoms.

More preferably, the aliphatic monoalcohol comprises 1 to 5 carbon atoms.

The most active one is methyl alcohol. However, ethyl alcohol and isopropyl, propyl, butyl, isobutyl and even amyl alcohols can be considered. Heavier alcohols such as ethyl-hexyl alcohol or lauric alcohol can also be used.

Methyl alcohol that facilitates the reaction can advantageously be added to the heavy alcohols.

Furthermore, when preparing the ethyl ester, it is possible to use a mixture of ethyl and methyl alcohol comprising 1 to 50 wt. %, preferably 1 to 10 wt. % methyl alcohol so as to increase the conversion.

Catalysts

Most of the catalysts encountered come in form of powders, balls, extrudates or pellets. These forming types remain valid in the case of porous hybrid solids such as those described in the present invention.

In cases where the reactor technology requires catalysts in form of balls, pellets, granules or extrudates, the various forming modes known to the person skilled in the art (see U.S. Pat. No. 6,893,564) can be used (impregnation, deposition, mixing-extrusion, granulation, pelletizing, etc.). The examples below illustrate in a non-exhaustive manner some of the methods that may be considered.

Coordination polymer powders can be subjected to granulation using, for example, organic or inorganic binders such as those described in patent application WO-2006/050,898.

Using binders, charges, peptizing agents furthermore allows catalysts to be formed as extrudates by mixing-extrusion.

The droplet coagulation technique can also be suitable for these hybrid solids.

Conventional methods of deposition on a suitable preformed support, or of impregnation or modification of a preformed support, well known to the person skilled in the art, can also be advantageously used.

All these forming types can be achieved in the presence or in the absence of a binder.

Alumina can for example be used as a binder. It allows to increase the surface area of the material and often to create a compound that is much more stable to leaching and mechanical stresses. Preferably, the alumina content represents up to 90 wt. % in relation to the total mass of the material formed. More preferably, the alumina content ranges between 10 and 70 wt. % in relation to the total mass of the material formed.

The coordination polymers consist of metal ions or of inorganic polyhedra of metal ions, or nodes, connected by polyfunctionalized organic molecules, or ligands, having at least two chelating functions (carboxylates, amines, phosphonates, sulfonates, alcoholates, etc.). These materials have pores, in particular micropores (size below 2 nm) and mesopores (size ranging between 2 and 50 nm). The specific surface areas of these materials can range from 5 to 5000 m²/g, preferably from 100 to 3000 m²/g.

Examples of metals making up the “nodes” of these materials are metals from groups 2 to 17 of the periodic table. In particular, metals such as Mg, Ca, Sr, Ba, Sc, Y, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, Hg, Al, Ga, in, Tl, Ge, Sn, Pb, As, Sb and Bi are preferably used. Among the latter, Zn, Cu, Cd, Ni, Fe, Co, Ru, Rh, Pd, Pt, Mn, Mg, Ag are preferred. By way of non limitative example, the metal ions present in the porous hybrid materials partly taken from the previous list are as follows: Mg²⁺, Ca²⁺, Sr²⁺, Ba²⁺, Sc³⁺, Y³⁺, Ti⁴⁺, Zr⁴⁺, Hf⁴⁺, V⁴⁺, V³⁺, V²⁺, Nb³⁺, Ta³⁺, Cr³⁺, Mo³⁺, W³⁺, Mn³⁺, Mn²⁺, Re³⁺, Re²⁺, Fe³⁺, Fe²⁺, Ru³⁺, Ru²⁺, Os³⁺, Os²⁺, Co³⁺, Co²⁺, Co⁺, Rh²⁺, Rh⁺, Ir²⁺, Ir⁺, Ni²⁺, Ni⁺, Pd²⁺, Pd⁺, Pt²⁺, Pt⁺, Cu²⁺, Cu⁺, Ag⁺, Au⁺, Zn²⁺, Cd²⁺, Hg²⁺, Al³⁺, Ga³⁺, In³⁺, Tl³⁺, Si⁴⁺, Si⁺, Ge⁴⁺, Sn⁴⁺, Sn²⁺, Pb⁴⁺, Pb²⁺, As⁵⁺, As³⁺, As⁺, Sb⁵⁺, Sb³⁺, Sb⁺, Bi⁵⁺, Bi³⁺, Bi⁺.

Preferably, the metal is selected from among groups 2 to 15 of the periodic table. More preferably, the metal is selected from among groups 2 and 7 to 12, and more particularly among Zn, Cu, Cd, Ni, Fe, Co, Ru, Rh, Pd, Pt, Mn, Mg, Ag.

By way of non limitative example, the metal ions present in the porous hybrid materials partly taken from the previous list are as follows: Mg²⁺, Ca²⁺, Sr²⁺, Ba², Sc³⁺, Y³⁺, Ti⁴⁺, Zr⁴⁺, Hf⁴⁺, V⁴⁺, V³⁺, V²⁺, Nb³⁺, Ta³⁺, Cr³⁺, Mo³⁺, W³⁺, Mn³⁺, Mn²⁺, Re³⁺, Re²⁺, Fe³⁺, Fe²⁺, Ru³⁺, Ru²⁺, Os³⁺, Os²⁺, Co³⁺, Co²⁺, Co⁺, Rh²⁺, Rh⁺, Ir²⁺, Ir⁺, Ni²⁺, Ni⁺, Pd²⁺, Pd⁺, Pt²⁺, Pt⁺, Cu²⁺, Cu⁺, Ag⁺, Au⁺, Zn²⁺, Cd²⁺, Hg²⁺, Al³⁺, Ga³⁺, In³⁺, Ti³⁺, Si⁴⁺, Si⁺, Ge⁴⁺, Sn⁴⁺, Sn²⁺, Pb⁴⁺, Pb²⁺, As⁵⁺, As³⁺, As⁺, Sb⁵⁺, Sb³⁺, Sb⁺, Bi⁵⁺, Bi³⁺, Bi⁺.

Examples of sources of metals that can be used are metal oxides and mixtures thereof in any proportion, as well as salts of these metals, halogenide, sulfate, nitrate, phosphate, carbonate, oxalate, hydroxide, alcoholate, perchlorate, carboxylate or acetylacetonate salts. These precursors can come in form of powder or formed, soluble or insoluble in the reaction medium.

The organic molecules having at least two chelating functions and making up the framework of the material can comprise an alkyl group with 1 to 10 carbon atoms, aryl groups (1 to 5 benzene rings), a mixture of alkyl groups (1 to 10 carbon atoms) and of aryl groups (1 to 5 benzene rings). These groups have to be functionalized by at least two chemical groups such as COOH, CS₂H, NO₂, NH₂, OH, SO₃H, Si(OH)₃, Ge(OH)₃, Sn(OH)₃, Si(SH)₃, Ge(SH)₃, Sn(SH)₃, PO₃H, AsO₃H, AsO₄H, P(SH)₃, As(SH)₃, CH(RSH)₂, C(RSH)₃, CH(RNH₂)₂, C(RNH₂)₃, CH(ROH)₂, C(ROH)₃, CH(RCN)₂, C(RCN)₃, where R is an alkyl group having between 1 and 10 carbon atoms or an aryl group having between 1 and 5 benzene rings, and CH(SH)₂, C(SH)₃, CH(NH₂)₂, C(NH₂)₃, CH(OH)₂, C(OH)₃, CH(CN)₂ and C(CN)₃. Besides, nitrogen-containing, sulfur-containing, oxygen-containing heterocycles, substituted or not, can also serve as ligands (pyridine, imidazole derivatives).

Ligands carrying carboxylic acid groups, substituted or not on the aromatic ring by the aforementioned groups, naphthalene dicarboxylate (NDC), or carrying amine groups such as bipyridines, are preferably used. More preferably, the organic ligand is terephthalic acid, substituted or not on the benzene ring or 2-methylimidazole.

More preferably, the porous hybrid solids with an organic-inorganic mixed matrix used as catalysts in the present invention consist of Zn²⁺ polyhedra or ions, and they are preferably connected by bidentate ligands derived from terephthalic acid.

Some methods for preparing these porous hybrid materials are known from the prior art and they are notably described in patents US-2006/0,287,190 or U.S. Pat. No. 7,196,210. The various synthesis paths leading to these solids are applicable within the scope of the present invention and the preparation modes presented here are in no way restrictive.

This type of catalyst can be advantageously prepared using one of the methods described hereafter.

A conventional method of preparing a coordination polymer comprises a first stage wherein the zinc precursor is brought into solution in water or in a polar organic solvent or a mixture of solvents, and the organic ligand is also brought into solution in water or in a polar organic solvent. In a second stage, these two solutions are mixed and stirred. A third stage consists in adding to this mixture a base in aqueous solution (methylamine for example) or in solution in a polar organic solvent. This final mixture is then stirred or not. The hybrid material precipitating in the medium, it is filtered, washed with water or with an organic solvent, then dried. It can be optionally subjected to a subsequent thermal treatment in order to clear the porosity.

A porous hybrid solid with an organic-inorganic mixed matrix preferably used as the catalyst in the present invention and consisting of Zn²⁺ polyhedra or ions connected by bidentate ligands derived from terephthalic acid is a hybrid crystallized material referred to as IHM-1, whose crystalline structure is detailed hereafter. Hybrid material IHM-1 has an X-ray diffraction diagram including at least the lines given in Table 1. This diffraction diagram is obtained by radiocrystallographic analysis using the conventional powder method with an X'Pert PRO PANalytical diffractometer equipped with a θ-θ goniometer, a copper X-ray tube (line Kα₁ at 1.5418 Å) provided with a rear monochromator. The material routine analyses were recorded with an 0.05° increment for 5 seconds, up to 70°. For more precise records, the increment is 0.02° for 10 seconds up to 120°.

From the position of the diffraction peaks shown by angle 2θ, the reticular distances dₕₖₗ characteristic of the sample are calculated by applying Bragg's relation. The measuring error Δ(dₕₖₗ) on dₕₖₗ is calculated according to the absolute error Δ(2θ) assigned to the measurement of 2θ. An absolute error Δ(2θ) equal to ±0.02° is commonly admitted. The relative intensity I/I₀ assigned to each value of dₕₖₗ is measured from the height of the corresponding diffraction peak. The X-ray diffraction diagram of hybrid material IHM-1 according to the invention comprises at least the lines for the values of dₕₖₗ given in Table 1. In the dₕₖₗ column, the mean values of the inter-reticular distances are given in Angströms (Å). Each one of these values has to be assigned the measuring error Δ(dₕₖₗ) ranging between ±0.3 Å and ±0.01 Å.

TABLE 1
Mean values of the dₕₖₗ and relative intensities measured in an X-ray
diffraction diagram of hybrid material IHM-1

| 2 Thêta (°) | dₕₖₗ (Å) | I/I₀ |
|---|---|---|
| 8.81 | 10.03 | FF |
| 14.22 | 6.22 | ff |
| 15.78 | 5.61 | f |
| 17.67 | 5.02 | m |
| 26.65 | 3.34 | ff |
| 27.11 | 3.28 | ff |
| 28.69 | 3.11 | f |
| 28.95 | 3.08 | f |
| 29.97 | 2.98 | ff |
| 30.51 | 2.93 | f |
| 31.11 | 2.87 | f |
| 31.90 | 2.80 | f |
| 32.55 | 2.75 | mf |
| 34.05 | 2.63 | ff |
| 34.97 | 2.56 | ff |
| 35.77 | 2.51 | f |
| 36.87 | 2.44 | f |
| 39.05 | 2.30 | ff |
| 40.39 | 2.23 | ff |
| 41.99 | 2.15 | ff |
| 42.75 | 2.11 | ff |
| 45.19 | 2.00 | f |

where FF=very high, F=high, m=medium, mf=medium low, f=low, ff=very low.
Intensity I/I₀ is given in relation to a relative intensity scale where a value of 100 is assigned to the line of highest intensity in the X-ray diffraction diagram: ff<15; 15≦f<30; 30≦mf<50; 50≦m<65; 65≦F<85; FF≧85.

This hybrid material IHM-1 is indexed by monoclinic symmetry with, as the cell parameters: a=20.21(7)Å; b=3.33(1)Å, c=6.28(6)Å and angles: α=γ=90° and β=97.1(4)°.

The method of preparing solid IHM-1 comprises the following stages:
i. dissolving at least one zinc precursor based on anhydrous zinc dichloride and terephthalic acid (H₂BDC) in at least one organic solvent
ii. bringing 2-methylamine (MEA) into solution in water
iii. optionally mixing the previous two solutions together
iv. crystallizing
v. filtering, washing and drying the product obtained.

The solvent involved in the synthesis contains in particular dimethylformamide (DMF). It can optionally be associated with toluene.

The crystallization stage is carried out between ambient temperature and 100° C. for 12 to 30 hours.

Drying is carried out between 40° C. and up to a temperature of 200° C. In most cases, drying is performed between 40° C. and 100° C., preferably between 45° C. and 75° C., for a duration ranging between 15 minutes and 1 hour, most often about 30 minutes. It is thereafter carried out between 100° C. and 200° C., preferably between 130° C. and 170° C., most often between 2 and 8 hours and usually for about 6 hours.

Operating Conditions of the Transesterification Reaction

The method is carried out at temperatures ranging between 130° C. and 220° C., at pressures below 100 bars, with excess monoalcohol in relation to the fatty substance/alcohol stoichiometry.

The reaction can generally be operated according to various embodiments.

If the reaction is carried out in discontinuous mode, it can be conducted in one or two stages, i.e. by carrying out a first reaction up to 85% to 95% conversion to esters, cooling by evaporating the excess alcohol, decanting the glycerin and ending the reaction by heating again to between 130° C. and 220° C. and by adding alcohol to obtain total conversion.

A 98% conversion to esters can also be aimed by working for a sufficiently long time in a single stage under suitable conditions, for example by increasing the temperature and/or the alcohol/fatty substance ratio.

If the reaction is carried out in continuous mode, it can be conducted with several autoclaves and decanters arranged in series. A partial conversion is performed in a first reactor, most often below 90%, generally at least 50% and in most cases approximately 85%, then decanting is achieved by evaporating the alcohol and by cooling; the transesterification reaction is completed in a second reactor under the aforementioned conditions by adding part of the alcohol previously evaporated. The excess alcohol is finally evaporated in an evaporator, and the glycerin and the esters are separated by decantation.

Thus, after these two stages, a biodiesel fuel meeting the specifications is obtained. The conversion level is adjusted so as to obtain an ester fuel meeting the specifications and a glycerin of high purity, by operating in one or two stages.

When selecting a fixed-bed continuous method, it can be advantageous to work at temperatures ranging between 130° C. and 220° C., preferably between 150° C. and 180° C., at pressures ranging between 10 and 70 bars, the LHSV preferably ranging between 0.1 and 3, more preferably between 0.3 and 2, in the first stage, and the alcohol/oil weight ratio ranging between 3/1 and 0.1/1.

Alcohol introduction can be advantageously fractionated. It can be fed into the tubular reactor at two levels as follows: supplying the reactor with the oil and about ⅔ of the alcohol involved, then supplying the rest of the alcohol approximately at the level of the upper third of the catalytic bed.

The leaching strength is verified in the present invention by the absence of traces from the catalyst, in the ester formed as well as in the glycerin produced.

The catalyst recyclability is experimentally evaluated over time.

If a temperature of 220° C. is not exceeded, an ester of same colour as the initial oil and a colourless glycerin are generally obtained after decantation.

Analysis of the compounds produced is performed either by gas chromatography for the esters and the glycerin or, more rapidly, by steric exclusion liquid chromatography for the esters.

The ester and the glycerol obtained contain no impurities from the catalyst. No purification treatment is therefore applied to eliminate the catalyst or residues thereof, unlike catalysts working according to a homogeneous process wherein the catalyst or its residues are, after the reaction, located in the same phase as the ester and/or the glycerin.

The reaction is thus conducted in one or two stages by adjusting the conversion level so as to obtain an ester fuel having a monoglyceride content of at most 0.8 mass %, a diglyceride content of at most 0.2 mass %, a triglyceride content of at most 0.2 mass % and a glycerin content of less than 0.25 mass %. The same procedure is applied to obtain a glycerin of purity ranging between 95 and 99.9%, preferably between 98 and 99.9%.

By means of this type of process, the final purification is reduced to a minimum while allowing to obtain an ester meeting the fuel specifications and a glycerin whose purity ranges between 95% and 99.9%, preferably between 98% and

99.9%.

### EXAMPLES

The following examples illustrate the invention without limiting the scope thereof, example 7 being given by way of comparison.

All the examples below were carried out in a closed reactor and they therefore correspond to a single stage. To obtain a biodiesel fuel meeting the specifications, it would be necessary to perform, at the end of this first stage, decantation by evaporating the alcohol and by cooling, then to complete the transesterification reaction by adding the evaporated alcohol part.

The oil used in these examples is rapeseed oil whose fatty acid composition is as follows:

TABLE 2
Rapeseed oil composition

| Fatty acid glyceride | Nature of the fatty chain | Mass % |
|---|---|---|
| Palmitic | C16:0 | 5 |
| Palmitoleic | C16:1 | <0.5 |
| Stearic | C18:0 | 2 |
| Oleic | C18:1 | 59 |
| Linoleic | C18:2 | 21 |
| Linolenic | C18:3 | 9 |
| Arachidic | C20:0 | <0.5 |
| Gadoleic | C20:1 | 1 |
| Behenic | C22:0 | <0.5 |
| Erucic | C22:1 | <1 |

However, any other oil of vegetable or animal origin could give similar results.

### Example 1

### Preparation of a Catalyst Based on a Hybrid Solid with an Organic-Inorganic Mixed Matrix IHM-1

A zinc precursor (ZnCl₂, purity>98%, Sigma) and terephthalic acid (H₂BDC, purity>98%, Sigma) are dissolved in 250 ml dimethylformamide (DMF, 99.8%, Sigma). The 2-methylamine (MEA, 40% in H₂O, Sigma) is brought into solution in 100 ml water and added to the previous mixture dropwise for 30 minutes. The reaction product is thereafter left to crystallize for 24 hours, then it is isolated through filtration and rinsed twice with DMF. The solid obtained is thereafter dried at 60° C. for 30 minutes, then at 150° C. for 6 hours.

Hybrid material IHM-1 thus obtained has an X-ray diffraction diagram involving at least the lines given in Table 1.

### Example 2

### Transesterification of Vegetable Oils (Rapeseed Oil) by Methanol from a Hybrid Solid Catalyst with an Organic-Inorganic Mixed Matrix IHM-1 at 200° C.

25 g rapeseed oil, 25 g methanol and 1 g catalyst IHM-1 prepared as described in Example 1 and in powder form are fed into a closed reactor at ambient temperature. The methanol/oil mass ratio is thus 1, which corresponds to a molar ratio of 27.5. The reactor is then closed, stirred (200 rpm) and heated to 200° C. by means of a heating magnetic stirrer. The temperature of the reaction medium is stabilized at 200° C. after 40 minutes heating. The pressure is the autogenous pressure of alcohol at the operating temperature. The reaction is monitored as soon as the temperature of the reaction medium has reached the set temperature value. Samples are regularly taken in order to follow the progress of the reaction. After 6 hours' reaction, stirring is stopped and the reactor is left to cool down to ambient temperature. The samples taken and the final effluent are washed by means of a NaCl-saturated aqueous solution then, after decantation, the upper organic phase is analysed by gel-permeation chromatography (GPC). The table hereafter shows the results obtained.

| | | Sampling (in h) | | | |
|---|---|---|---|---|---|
| | | 0^{b} | 2 | 4 | 6 |
| % | Triglycerides | 20 | 1 | 0.2 | 0.4 |
| in the organic | Diglycerides^{c} | 20 | 3 | 2 | 2 |
| phase^{a} | Monoglyceride | 14 | 6 | 5 | 6 |
| | Vegetable oil methyl esters | 46 | 89 | 92 | 92 |

^{a}determined by GPC
^{b}t = 0 when the reaction medium is at temperature
^{c}% representing the diglycerides and sterols

Conversion of the triglycerides starts even though the reaction medium has not reached 200° C. (46% esters at t0). The conversion (estimated in relation to the triglycerides, conversion=1−m_{final} (triglycerides)/mᵢₙᵢₜᵢₐₗ (triglycerides)), is 99% in 120 minutes.

Leaching of the catalyst in the ester phase is negligible (the zinc content estimated by means of the inductively coupled plasma (ICP) technique is below 200 ppm). This result is valid for all the examples below.

### Example 3

### Transesterification of Vegetable Oils (Rapeseed Oil) by Methanol from a Hybrid Solid Catalyst with an Organic-Inorganic Mixed Matrix IHM-1 at 180° C.

Example 2 is repeated using 25 g rapeseed oil, 25 g methanol and 1 g catalyst IHM-1 prepared according to Example 1 and in powder form. The reaction is carried out at 180° C., the temperature of the reaction medium being stabilized at 180° C. after 20 minutes heating. The table below gives the results obtained.

| | | Sampling (in h) | | | | |
|---|---|---|---|---|---|---|
| | | 0^{b} | 0.33 | 0.66 | 1 | 2 |
| Mass % in | Triglycerides | 49 | 16 | 6 | 4 | 1 |
| the | Diglycerides^{c} | 24 | 18 | 11 | 8 | 3 |
| organic | Monoglyceride | 7 | 15 | 15 | 13 | 9 |
| phase^{a} | Vegetable oil | 20 | 50 | 67 | 75 | 86 |
| | methyl esters | | | | | |

^{a}determined by GPC
^{b}t = 0 when the reaction medium is at temperature
^{c}% representing the diglycerides and sterols

Conversion of the triglycerides starts even though the reaction medium has not reached 180° C. (20% esters at t0). The conversion (estimated in relation to the triglycerides) is 99% in 120 minutes.

### Example 4

### Transesterification of Vegetable Oils (Rapeseed Oil) by Methanol from a Hybrid Solid Catalyst with an Organic-Inorganic Mixed Matrix IHM-1 at 160° C.

Example 2 is repeated using 25 g rapeseed oil, 25 g methanol and 1 g catalyst prepared according to Example 1 and in powder form. The reaction is carried out at 160° C., the temperature of the reaction medium being stabilized at 160° C. after 20 minutes heating. The table below gives the results obtained.

Sampling (in h)

| | | 0^{b} | 2 | 4 | 6 |
|---|---|---|---|---|---|
| Mass % in | Triglycerides | 82 | 8 | 2 | 1 |
| the | Diglycerides^{c} | 12 | 12 | 6 | 4 |
| organic | Monoglyceride | 1 | 15 | 12 | 9 |
| phase^{a} | Vegetable oil | 5 | 65 | 81 | 86 |
| | methyl esters | | | | |

^{a}determined by GPC
^{b}t = 0 when the reaction medium is at temperature
^{c}% representing the diglycerides and sterols

The conversion (estimated in relation to the triglycerides) is 99% in 6 hours.

### Example 5

### Preparation of a Catalyst Based on a Hybrid Solid with an Organic-Inorganic Mixed Matrix

A methanoic 2-methylimidazole solution (1.642 g in 50 ml MeOH) is fed dropwise, under stirring, into an ammoniacal Zn(OH)₂ solution (0.994 g in 100 ml NH₃ 25%). After introducing all of the methanoic solution, stirring is stopped and the solid is left to precipitate for 4 days. The solid is thereafter filtered and washed with 3*50 ml of an H₂O/MeOH solution (1:1 v:v), then dried in the open air (X-C Huang, et al., Angew. Chem. Int. Ed., 2006, 45, 1557-1559).

### Example 6

### Transesterification of Vegetable Oils (Rapeseed Oil) by Methanol from a Hybrid Porous Solid Catalyst with an Organic-Inorganic Mixed Matrix at 180° C.

Example 2 is repeated using 25 g rapeseed oil, 25 g methanol and 1 g catalyst prepared according to Example 5 and in powder form. The reaction is carried out at 180° C., the temperature of the reaction medium being stabilized at 180° C. after 20 minutes heating. The table below gives the results obtained.

| | | Sampling (in h) | | | | |
|---|---|---|---|---|---|---|
| | | 0^{b} | 0.33 | 1 | 2 | 4 |
| Mass % | Triglycerides | 65 | 28 | 7 | 1 | 0 |
| in the | Diglycerides^{c} | 19 | 23 | 11 | 6 | 3 |
| organic | Monoglyceride | 4 | 13 | 15 | 12 | 7 |
| phase^{a} | Vegetable oil | 12 | 36 | 67 | 79 | 90 |
| | methyl esters | | | | | |

^{a}determined by GPC
^{b}t = 0 when the reaction medium is at temperature
^{c}% representing the diglycerides and sterols

The conversion (estimated in relation to the triglycerides) is 99% in 2 hours.

### Example 7 (Comparative)

### Transesterification of Rapeseed Oil by Methanol in the Presence of zinc aluminate (ZnAl₇O₄) in Powder Form at 200° C.

Example 2 is repeated using 25 g rapeseed oil, 25 g methanol and 1 g catalyst ZnAl₂O₄ in powder form. The reaction is carried out at 200° C., the temperature of the reaction medium being stabilized at 200° C. after 40 minutes heating. The table below gives the results obtained.

| | | Sampling (in h) | | | |
|---|---|---|---|---|---|
| | | 0^{b} | 2 | 4 | 6 |
| Mass % | Triglycerides | 71 | 26 | 6 | 1 |
| in the | Diglycerides^{c} | 15 | 15 | 6 | 3 |
| organic | Monoglyceride | 2 | 9 | 9 | 8 |
| phase^{a} | Vegetable oil | 12 | 49 | 80 | 88 |
| | methyl esters | | | | |

^{a}determined by GPC
^{b}t = 0 when the reaction medium is at temperature
^{c}% representing the diglycerides and sterols

This result clearly shows that zinc aluminate catalyzes the transesterification reaction much more slowly than a hybrid solid with an organic-inorganic mixed matrix since the performances at 200° C. are equivalent to those of the coordination polymer at lower temperature (180° C. in Example 6).

1) A method of preparing a composition of alcohol esters of linear monocarboxylic acids with 6 to 26 carbon atoms and glycerin, wherein a fatty substance of animal or vegetable origin is reacted with an aliphatic monoalcohol having 1 to 18 carbon atoms, in the presence of at least one heterogeneous catalyst based on a hybrid solid with an organic-inorganic mixed matrix consisting of metal ions or of metal ion polyhedra connected to one another by at least one at least bidentate polyfunctionalized organic ligand.

2) A method as claimed in claim 1, wherein said aliphatic monoalcohol comprises 1 to 12 carbon atoms.

3) A method as claimed in claim 1, wherein the alcohol involved is a mixture of ethyl and methyl alcohol comprising 1 to 50 wt. %, preferably 1 to 10 wt. % methyl alcohol.

4) A method as claimed in claim 1, wherein the method is carried out at a temperature ranging between 130° C. and 220° C., at a pressure below 100 bars and with excess monoalcohol in relation to the fatty substance/alcohol stoichiometry.

5) A method as claimed in claim 1, wherein the initial oil is selected from among the following oils: palm oil (concrete or olein), soybean oil, palm nut oil, copra oil, babassu oil, rapeseed oil, old or new, sunflower oil, conventional or oleic, corn oil, cotton oil, peanut oil, pourgher oil, castor oil, linseed oil and crambe oil, algae oil and the sunflower or rapeseed oils obtained by genetic engineering or hybridization, oils partly modified by polymerization or oligomerization, waste kitchen oil, slaughterhouse oil, fish oil, seal oil, tallow, lard, fat from sewage treatment.

6) A method as claimed in claim 1, characterized in that the catalyst comes in form of powder, extrudates, balls or pellets.

7) A method as claimed in claim 1, wherein alumina is used as the binder in proportions up to 90 wt. % of the total mass of the material formed.

8) A method as claimed in claim 1, wherein the metal ion is selected from among metals of groups 2 to 17 of the periodic table, preferably among Zn, Cu, Cd, Ni, Fe, Co, Ru, Rh, Pd, Pt, Mn, Mg, Ag.

9) A method as claimed in claim 1, wherein the bidentate organic ligand comprises an alkyl group with 1 to 10 carbon atoms, an aryl group with 1 to 5 benzene rings or a mixture thereof, these groups being functionalized by at least two chemical groups selected from among COOH, CS₂H, NO₂, NH₂, OH, SO₃H, Si(OH)₃, Ge(OH)₃, Sn(OH)₃, Si(SH)₃, Ge(SH)₃, Sn(SH)₃, PO₃H, AsO₃H, AsO₄H, P(SH)₃, As(SH)₃, CH(RSH)₂, C(RSH)₃, CH(RNH₂)₂, C(RNH₂)₃, CH(ROH)₂, C(ROH)₃, CH(RCN)₂, C(RCN)₃, where R is an alkyl group having between 1 and 10 carbon atoms or an aryl group having between 1 and 5 benzene rings, and CH(SH)₂, C(SH)₃, CH(NH₂)₂, C(NH₂)₃, CH(OH)₂, C(OH)₃, CH(CN)₂ and C(CN)₃.

10) A method as claimed in claim 9, wherein the bidentate organic ligand is selected from among nitrogen-containing, sulfur-containing, oxygen-containing heterocycles, substituted or not.

11) A method as claimed in any one of claim 9 or 10, claim 9, wherein the organic ligand is terephthalic acid substituted or not on the benzene ring.

12) A method as claimed in claim 9, wherein the organic ligand is 2-methylimidazole.

13) A method as claimed in claim 1, wherein the heterogeneous catalyst based on a hybrid solid with an organic-inorganic mixed matrix consists of Zn²⁺ metal ions or metal ion polyhedra connected to one another by at least one organic ligand of terephthalic acid type.

14) A method as claimed in claim 13, wherein said catalyst is material IHM-1 having an X-ray diffraction diagram including at least the lines given in the table below, and indexed by monoclinic symmetry with, as the cell parameters: a=20.21(7)Å; b=3.33(1)Å, c=6.28(6)Å and angles: □=□=90° and □=97.1(4)°:

| 2 Thêta (°) | dₕₖₗ (Å) | I/I₀ |
|---|---|---|
| 8.81 | 10.03 | FF |
| 14.22 | 6.22 | ff |
| 15.78 | 5.61 | f |
| 17.67 | 5.02 | m |
| 26.65 | 3.34 | ff |
| 27.11 | 3.28 | ff |
| 28.69 | 3.11 | f |
| 28.95 | 3.08 | f |
| 29.97 | 2.98 | ff |
| 30.51 | 2.93 | f |
| 31.11 | 2.87 | f |
| 31.90 | 2.80 | f |
| 32.55 | 2.75 | mf |
| 34.05 | 2.63 | ff |
| 34.97 | 2.56 | ff |
| 35.77 | 2.51 | f |
| 36.87 | 2.44 | f |
| 39.05 | 2.30 | ff |
| 40.39 | 2.23 | ff |
| 41.99 | 2.15 | ff |
| 42.75 | 2.11 | ff |
| 45.19 | 2.00 | f |

where FF=very high, F=high, m=medium, mf=medium low, f=low, ff=very low. Intensity I/I₀ is given in relation to a relative intensity scale where a value of 100 is assigned to the line of highest intensity in the X-ray diffraction diagram: ff<15; 15≦f<30; 30≦mf<50; 50≦m<65; 65≦F<85; FF≧85.

15) A method as claimed in claim 1, wherein the hybrid solid with an organic-inorganic mixed matrix has a BET specific surface area ranging between 5 and 5000 m²/g.

16) A method as claimed in claim 1, wherein the reaction is carried out in discontinuous mode.

17) A method as claimed in claim 1, wherein the method is carried out in continuous mode, with a fixed bed or autoclaves and decanters arranged in series.

18) A method as claimed in claim 17, wherein the reaction is carried out in a fixed bed, at a pressure ranging between 10 and 70 bars and at a LHSV ranging between 0.1 and 3, with an alcohol/fatty substance weight ratio ranging between 3/1 and 0.1/1.

19) A method as claimed in claim 1, characterized in that it is carried out in one or two stages by adjusting the conversion level so as to obtain an ester fuel having a monoglyceride content of at most 0.8 mass %, a diglyceride content of at most 0.2 mass %, a triglyceride content of at most 0.2 mass % and a glycerin content of less than 0.25 mass %.

20) A method as claimed in claim 1, characterized in that it is carried out in one or two stages by adjusting the conversion level so as to obtain a glycerin of purity ranging between 95 and 99.9%, preferably between 98 and 99.9%.
